(19) United States
(12) Patent Application Publication
(10) Pub. No.: US 2015/0152338 A1
(43) Pub. Date: Jun. 4, 2015

(54) METHOD FOR REDUCING QUENCH OIL FOULING IN CRACKING PROCESSES

(71) Applicant: BAKER HUGHES INCORPORATED, Houston, TX (US)

(72) Inventors: Marco Respini, Casalmorano (IT); Enrico Madeddu, Cagliari (IT)

(73) Assignee: BAKER HUGHES INCORPORATED, Houston, TX (US)

(21) Appl. No.: 14/616,145

(22) Filed: Feb. 6, 2015

Related U.S. Application Data
(63) Continuation-in-part of application No. 12/024,251, filed on Feb. 1, 2008, now abandoned.
(60) Provisional application No. 60/888,466, filed on Feb. 6, 2007.

(51) Int. Cl. C10G 9/00; G01N 21/49; G01N 33/28; C07C 4/00; C10B 39/00

(52) U.S. Cl. CPC C10G 9/00; C07C 4/00; C10B 39/00; G01N 33/2841; G01N 21/49

(57) ABSTRACT

Quench oil aging and its propensity to cause fouling may be evaluated by determining the amount of a precipitant necessary to cause the flocculation of polymer species present in the quench oil. The propensity of quench oil to cause fouling may be used as a basis to mitigate fouling in cracking processes.

### CROSS-REFERENCE TO RELATED APPLICATION

This application is a Continuation-in-Part and claims priority to U.S. application Ser. No. 12/024,251 filed on Feb. 1, 2008; which claims priority to U.S. Provisional Application Ser. No. 60/888,466 filed on Feb. 6, 2007; all of which are incorporated by reference herein in their entirety.

### TECHNICAL FIELD

The present invention relates to a method for reducing fouling in cracking processes. The present invention particularly relates to a method for reducing fouling from quench oil in cracking processes due to aging of the quench oil.

### BACKGROUND

Petrochemical plants, which include both Chemical Production Installations as well as Oil Refineries, are known to employ two basic types of furnaces. The first of these is a steam cracker furnace. Steam crackers are used in applications including the production of ethylene. The second of these is a “steam reformer” furnace, which can be used to make hydrogen. Both types of furnaces include a number of tubes, generally arranged vertically, that form a continuous flow path, or coil, through the furnace. The flow path or coil includes an inlet and an outlet. In both types of furnaces, a mixture of a hydrocarbon feedstock and steam are fed into the inlet and passed through the tubes. The tubes are exposed to extreme heat generated by burners within the furnace. As the feedstock/steam mixture is passed through the tubes at high temperatures the mixture is gradually broken down such that the resulting product exiting the outlet is ethylene in the case of a steam cracker furnace and hydrogen in the case of a steam reformer furnace as well as other products including gasoline and coke.

During the cracking processes, the feed materials are heated to very high temperatures, in some embodiments, up to 900° C. This output is cooled by mixing it with a colder fluid and fed in a fractionating column where the separation of ethylene and light gasoline from a heavier oil takes place. The quality of the distillation, i.e. the amount of ethylene, light olefins and gasoline extracted from the top of the column, may be influenced by the temperature of the feed in the fractionating column. A higher temperature results in a higher yield of light products, which is often desirable. Attempting to handle such hot materials is usually not desirable and thus the need for a cooling step.

In some processes, the cooling step is implemented by admixing the very hot products from the cracking units with a comparatively cool fluid. The cool fluid, often an oil and most often a heavy oil, is typically referred to in the art as a “quench oil.” The heavy quench oil may be extracted from the process and is marketable as fuel oil.

In many processes, a minor amount of the quench oil is extracted to be used as a fuel, while the remaining part is recycled, sometimes back into the cracking process as a feed to the cracking unit or as reuse as a quench oil or both. During the course of its use, the heavy oil which is used as a quench oil may be continually exposed to temperatures ranging from 100 to 220° C. for extended periods of time.

Recycling quench oil may result in a number of serious unfavorable side effects. For example, viscosity increases of the recycled quench oil may be observed. In fact, the recirculating quench oil may remain in the circuit at relatively high temperatures for long periods of time, and this causes its aging. Symptomatic of this aging is the presence of unsaturated compounds, polymer and rubber formation, and a resulting viscosity increase. All of these side effects obviously may cause a negative impact upon the functioning of a production plant. Such negative impacts include an increase in the power required by the recirculation pumps, a reduction of the thermal exchange coefficients involved in steam production, and an increase of the maintenance costs involved in the cleaning of the plant components exposed to the quench oil.

### SUMMARY

In one aspect the invention is a method for reducing fouling from quench oil comprising treating a hydrocarbon feed using a cracking process having a quenching step, wherein: quench oil used in the quenching step has a known tendency to cause fouling; and the known tendency of the quench oil to cause fouling has been determined by measuring a tendency of the quench oil to precipitate polymeric species.

In another aspect the invention is method for reducing fouling from quench oil comprising treating a hydrocarbon feed using a cracking process having a quenching step, wherein process conditions in the cracking process have been adjusted based upon the tendency of quenching oil in the quenching step to cause fouling which is determined by measuring the tendency of the quench oil to precipitate polymeric species.

In one aspect the invention is a method for reducing fouling from quench oil in a cracking process comprising treating a hydrocarbon feed using a cracking process having a quenching step, introducing an additive to reduce fouling to the cracking process based upon a tendency of the quench oil in the quenching step to cause fouling which is determined by measuring a tendency of the quench oil to precipitate polymeric species.

In another aspect, the invention is a method for predicting the tendency for a quench oil to cause fouling in a cracking process by measuring the tendency of the quench oil to precipitate polymeric species.

In still another aspect, the invention is a method for measuring the tendency of the quench oil to precipitate polymeric species.

In another aspect, the invention is an apparatus for measuring the tendency of the quench oil to precipitate polymeric species.

### BRIEF DESCRIPTION OF THE DRAWING

For a detailed understanding of the present invention, reference should be made to the following detailed description of the preferred embodiments, taken in conjunction with the accompanying drawing(s) wherein:

FIG. 1 is graph showing the typical output of a transmittance probe in a quench oil sample during the addition of a precipitant to the quench oil sample.

### DETAILED DESCRIPTION

In a non-limiting embodiment, fouling from quench oil may be reduced in a cracking process comprising treating a hydrocarbon feed using a cracking process having a quenching step. Cracking processes are well known in the art of refining oil and other chemical processes. Such processes include, but are not limited to those disclosed in U.S. Pat. Nos. 6,096,188; 5,443,715; and 5,215,649; which are fully incorporated herein by reference. In another non-limiting embodiment, a quench oil is contacted with an intermediate or even a final product of a cracking process.

The quench oils may be or include, but are not limited to, crude oil; the precursors of naphthalene, phenanthrene, pyrene, quinoline, and hydroquinone; alkyl derivatives of naphthalene, phenanthrene, pyrene, quinoline, and hydroquinone. The quench oils may also be selected from the group consisting of aromatic molecules containing phenol groups and aromatic molecules containing non-phenolic oxygen substitutes. Also useful as the quench oil in some non-limiting embodiments are those compounds selected from the group consisting of steam cracked quench oils, steam cracked tars, cat cracked tars, cat cracked cycle oils, cat cracked bottoms, coker gas oils, coal tar oils, and aromatic extent oils and cuts of steam cracked quench oils, steam cracked tars, cat cracked tars, cat cracked cycle oils, cat cracked bottoms, coker gas oils, coal tar oils, and aromatic extract oils.

The hydrocarbons feeds that can be treated may be or include, but are not limited to, crude oil and intermediate refinery products resulting from the refining of crude oil.

In the process of treating a hydrocarbon feed using a cracking process, many products may be made including ethylene, gasoline, diesel fuel, other fuel oils, and coke. Processes producing heavy oils and coke are often subject to fouling. For the purposes of this application, fouling is a condition wherein materials having a very high viscosity and mixtures of viscous materials and solids such as coke deposits from the quench oil and accumulate within process equipment causing reduced operational efficiency or even shutting down the processing equipment. For example, when fouling occurs, it may cause transfer pipes to clog, which in turn may require the cracking unit to reduce process throughput or even shut down the unit. Such slow-downs and shut-downs often result in increased operating costs for the units affected and also any integrated units upstream or downstream of the affected unit.

In one non-limiting embodiment, the tendency to produce fouling of a quench oil is determined by measuring the tendency of the quench oil to precipitate polymeric species. Stated another way, the difference in solubility parameters of candidate quench oils for use in a cracking process and for polymeric species present therein can be measured and this measurement used as a basis for evaluating the propensity of the quench oil to undergo a polymer phase separation which may cause the deposition of foulants during a cracking process.

In a non-limiting embodiment, the polymeric species, also known as foulants, may be or include coke, asphaltene, polynuclear aromatic hydrocarbons, coke precursors, and combinations thereof. The polynuclear aromatic hydrocarbons may be or include, but are not limited to, asphaltenes, coke, coke precursors, naphthalene, perylene, coronene, chrysene, anthracene, and combinations thereof.

The tendency of candidate quench oils to precipitate polymeric species may be determined by any means known to those of ordinary skill in the art of making such determinations to be useful.

In a non-limiting embodiment, at least one parameter of the quench oil may be measured prior to selecting the quench oil, such as but not limited to, an insolubility number, a solubility blending number, and combinations thereof.

To measure the stability of the polymeric species therein, a first refractive index (RI) measurement may be taken with a refractive index probe inserted into the quench oil when the quench oil is undiluted, i.e. the quench oil does not include a solvent and/or precipitant. The first RI measurement may be used to determine a first functional refractive index (F_{RI}) value by using the formula F_{RI}=(RI²−1)/(RI²+2) where RI is the first refractive index measurement in this instance. The first F_{RI} value may determine a first solubility parameter, also known as a solubility blending number (SBn), by using the formula δ<52.042F_{RI}+2.904 (2) where δ is in units of 0.5 MPa where a linear correlation between the solubility parameter, δ, and FRI at 20° C. may be established.

This correlation was established based on the one-third rule relating to the function of the refractive index divided by the mass density as a constant equal to ⅓ for all different compounds. This rule was validated on more than 229 crude oils at 20° C. as well as higher temperatures up to 80° C.

U.S. patent application Ser. No. 13/924,089 filed Jun. 22, 2012 discusses RI parameters measured online using a refractive index probe to convert the RI values into a “solubility blending number” (SBn) based on a linear correlation. The linear correlation may be established using any method known in the art, such as, for example, that disclosed in the method published by the New Mexico Petroleum Recovery Research Center as PRRC 01-18. This document, authored by Jianxin Wang and Jill Buckley and having the title: Procedure for Measuring the Onset of Asphaltenes Flocculation.

A second refractive index (RI) measurement may be taken with a refractive index probe inserted into the quench oil stream during a turbidimetric flocculation titration, i.e. the quench oil undergoes a series of dilutions with a solvent and/or precipitant to induce polymeric species precipitation. An RI measurement may be taken at each dilution with the solvent or precipitant; each RI measurement may be converted into F_{RI} values and subsequent solubility blending numbers. At the point when the quench oil begins precipitating polymeric species, also known as polymeric species flocculation, another RI measurement may be taken to determine another F_{RI} value and thereby determine another solubility blending number. The solubility blending numbers may be plotted on a graph where the RI measurement is plotted on the x-axis, and the solubility blending number corresponding to each RI measurement is plotted on the y-axis. The slope of the graph is the insolubility blending number of the quench oil.

Obtaining the first solubility parameter and second solubility parameter may occur for a plurality of quench oils, and the first solubility parameter and second solubility parameter of a first quench oil may be compared to each quench oil within the plurality of quench oils. Based on the ratio of the first solubility parameter to the second solubility parameter for each quench oil, a quench oil may be selected from the plurality of quench oils where the selected quench oil has the smallest tendency for fouling. In a non-limiting embodiment, the first and second solubility parameters for a particular quench oil may be measured over a period of time.

For example, in one non-limiting embodiment, a sample of a quench oil candidate is placed in a container with a probe capable of measuring light scattering properties of the quench oil. In this embodiment, aliquots of a precipitant are added to the quench oil and the light scattering properties of the quench oil measured. A precipitant having a high light transmission level relative to the quench oil is used and the “dilution” effect of the precipitant will initially cause a reduction of light scattering in the sample until sufficient precipitant is added to the sample to cause precipitation of the polymer species thereby increasing light scatter. By comparing the amount of precipitant required to cause an increase in light scattering, sometimes also referred to as flocculation, quench oil candidates may be compared. In one non-limiting embodiment, quench oil candidates requiring more precipitant to increase light scattering are considered less likely to foul than those candidates requiring less precipitant.

A three dilution approach may be used. Quench oil samples of known amounts may be diluted at three different ratios: 1:1, 1:2, 1:1.5, and so on until polymeric species begin precipitating from the quench oil sample in a non-limiting embodiment. At each dilution, a refractive index measurement may be taken, and the refractive index measurement may be plotted on the x-axis, and its respective SBn value may be plotted on the y-axis.

Precipitants may be or include any precipitants that have a higher light transmission than the quench oil samples to be tested and which will cause a precipitation of polymer species from the quench oil. In one embodiment, these precipitants are selected from aliphatic solvents. Typical aliphatic solvents may be or include, but are not limited to, pentane, hexane, heptane, octane, isobutane, cyclohexane, and the like. Any precipitant may be used as long as it meets the specified criteria.

It may be desirable to dilute the quench oil with a solvent in a non-limiting embodiment. For example, in the case of colored quench oil candidates, it may be desirable to dilute the quench oil candidates to a point that they are within a specified transmission scale for a particular type of probe. The solvents used should be selected so that they do not materially interfere with the precipitation of polymeric species. For example, in one non-limiting embodiment, the solvents may be aromatic solvents. Such solvents include, but are not limited to benzene, toluene, xylene, ethyl benzene, and mixtures thereof.

Once the amount of precipitant necessary to cause onset of flocculation is known, it may be desirable to repeat the experiment with differing amounts of solvent and determine the flocculation point by means of a linear regression calculation. Any method of comparing the results from the measurements may be used to evaluate the relative propensity of various quench oil candidates to precipitate polymer species.

In one non-limiting embodiment, an automatic titrator is used in conjunction with a light probe to determine the flocculation point of a quench oil. An automatic titrator advantageously can dispense exact aliquots of precipitants and, when networked with suitable equipment, also record light scattering of sample therein. In an alternative embodiment, the automatic titrator, probe, and other equipment are networked to a controller. In many such embodiments, the controller is a personal computer.

The flocculation point of a quench oil is determined in some non-limiting embodiments by noting the point at which during a series of addition of precipitant to a quench oil sample, that light scattering starts to increase. The ability of a sample of quench oil to scatter light may be measured by any means known to useful to those of ordinary skill in the art of making such measurements. Preferably, the measurement is made using a probe and most preferably using a fiber optic probe. Exemplary fiber optic probes include transmission probes, reflectance probes, and attenuated total reflectance probes. Each of these probes has strengths and weaknesses that would make them more or less desirable for any given set of conditions. Those of ordinary skill in the art of making such measurements will know which probe to select for an application. For example, where the sample have a high level of opacity, it may be more desirable to use an attenuated total reflectance probe rather than a transmission probe. In one preferred embodiment, a fiber optics diffuse reflectance probe is used wherein a single fiber acts as a light source and 6 other fibers arranged around the source collect backscattered light.

The type of light employed by each probe may also be selected according to the conditions of the desired testing conditions. For example, the light employed may be UV, VIS or NIR. Such probes often employ silicon or germanium detectors. Any device useful for measuring light intensity may be used.

The type of probe used will determine whether flocculation is observed by a decrease or an increase in light intensity at a detector. As a sample increases in ability to scatter light, less light passes directly through the sample. Transmittance probes function by measuring the amount of light passing through a sample. Using a transmittance probe, an increase in the power of the light reaching the detector may occur until the flocculation point at which time the power may rapidly decrease. For a reflectance probe, the observations would be the inverse with power decreasing until the flocculation point.

In addition to making single determinations, the method may be used continuously. In this non-limiting embodiment, the flocculation point of recycled quench oil may be measured as a function of time. As the amount of precipitant need to cause flocculation decreases, the likelihood of fouling increases. At some point in time, either based upon prior experience or use of a predictive model, the determined tendency of the recycled quench oil to foul is used as a basis to divert the quench oil from recycle to an alternative disposition such as use as a fuel oil or the like. In an alternative non-limiting embodiment, rather than diverting quench oil as it reaches a certain tendency to foul, the process parameters may be changed to slow or prevent quench oil “aging.” For the purposes of the present application, “quench oil aging” means the phenomena where quench oil has a greater tendency to foul with time held at high temperatures such as is observed with quench oil that has been recycled. In still another non-limiting embodiment, the measured tendency of the quench oil to foul can be used as a basis for a decision to introduce additives into the cracking process to reduce fouling.

Additives useful for quench oil viscosity fouling reduction and control include, but are not limited to, well known chemistries to those skilled in the art, such as dispersants, radical scavengers and fouling control additives made of overbased metal carboxylates and sulphonates. To further reduce fouling in or from the quench oil, an antifoulant may be introduced into the quench oil or hydrocarbon feed, such as but not limited to, commercial dispersant/antifoulant product BPR34260 supplied by Baker Petrolite Corporation, antioxidants based on sterically hindered phenols and phenols, and their blends with amines such phenylene diamine and magnesium oxide overbase.

The density, type and opacity of a particular quench oil to be evaluated may determine how the quench oils will be tested. Those of ordinary skill in the art of running a cracking unit are knowledgeable regarding the methodology necessary to test their processes. Still, generally, samples tested according to the method may have sample sizes running from about 3 grams to about 50 grams. When diluted, the quench oils may be diluted in ratios (quench oil: Aromatic solvents) ranging from about 10:1 to about 1:20, and in some embodiments from about 2:1 to about 1:3. Typically, samples of quench oil are heated to from about 45 to about 60° C. prior to testing.

In an alternative non-limiting embodiment, Hildebrand solubility parameters are determined for a sample of quench oil. The Hildebrand solubility parameters are determined by making several runs with the quench oil dissolved in varying amounts of aromatic solvent. The quantity of precipitant needed to reach the flocculation point is divided by the sample size of the quench oil and linearly correlated with the dilution ratio. From this relationship, the Hildebrand solubility parameters are then determined.

In some non-limiting embodiments, it may be desirable to adjust process conditions in the cracking process based upon the tendency of quenching oil in the quenching step to cause fouling. While those of ordinary skill in the art are well aware of how to adjust a specific cracking process based upon a understanding of whether or not the quench oil used in the cracking process is likely to cause fouling, generally process parameters that could be adjusted include process temperatures, pressures, and residence times. For example, in at least some cracking processes, if an operator of the cracking process was aware that the quench oil used in the cracking process was likely to cause fouling, the operator may elect to decrease residence times, lower cracking temperatures, or increase pressures within the cracking process. In other embodiments, an operator may select to make the same or different adjustments based upon the specific characteristics of the subject cracking process. In one specific example, an operator may elect to change quench oil column (also known as Pyrolysis Column) bottom temperature, bottom column level, and rate of reflux of pyrolysis gasoline to the quench oil column.

While not wishing to be bound by any theory, it is believed that the polymer species that is precipitated from quench oils that result in the deposition of foulants within a cracking process are heavy aromatic polymers.

### EXAMPLES

The following examples are provided to illustrate the present invention. The examples are not intended to limit the scope of the present invention and they should not be so interpreted. Amounts are in weight parts or weight percentages unless otherwise indicated.

### Example 1

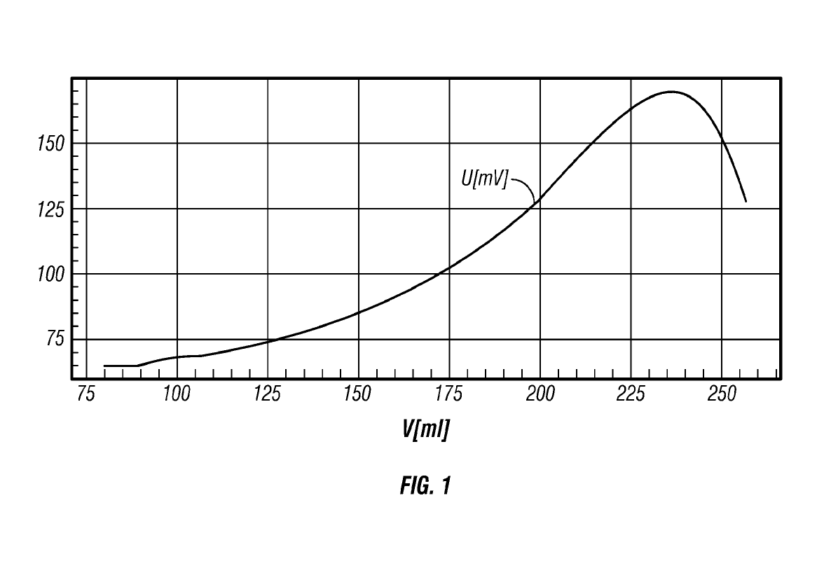

A sample of quench oil is placed into an automatic titrator. The reservoir of the automatic titrator is filled with normal heptane. A transmission probe is placed into contact with the quench oil sample and both the transmission probe and the automatic titrator are attached to a controller that records both light scattering and ml of n-heptane introduced into the sample. A curve showing a plot of this experiment is displayed in FIG. 1.

### Example 2

Five quench oil candidate materials are tested on an apparatus substantially identical to that of Example 1. Each material is tested 5 times and the data compared. For each sample, the repeatability of flocculation point is less than 3 percent of the precipitant used.

### Example 3 (Hypothetical)

The samples tested in Example 2 are evaluated for use with a steam cracker unit. The samples have a comparative value for flocculation point of:

Sample I: 1.2

Sample II: 2.9

Sample III: 1.7

Sample IV: 1.7

Sample V: 1.0

Sample II is selected as the quench oil for the unit.

### Example 4 (Hypothetical)

The recycle quench oil is tested substantially identically to Example 1 except that samples are removed from a cracking unit every 12 hours. The rate in decrease of the flocculation point is measured and compared against known conditions resulting in increased fouling. When the flocculation point decreases to the point that increased fouling appears likely to occur, the recycle quench oil is diverted for alternative disposition.

### Example 5 (Hypothetical)

Example 4 is repeated substantially identically except that instead of diverting the quench oil from recycle, additives are introduced into the cracking unit to reduce fouling.

### Example 6 (Hypothetical)

Example 4 is repeated substantially identically except that instead of diverting the quench oil from recycle, the conditions in the cracking unit are adjusted to extend the useful life of the quench oil.

### Discussion of the Examples

Example 1 and FIG. 1 clearly show that from the beginning of the experiment until about 23.5 ml of precipitant had been introduced into the sample, light transmission increased, caused by the dilution effect of the precipitant. At about 23.5 ml, scattering stopped decreasing and began increasing. This is the point at which flocculation occurred.

What is claimed is:

1. A method for reducing fouling from a quench oil comprising:
obtaining at least one measurement related to light scattering properties for at least one quench oil with a probe; wherein the at least one quench oil comprises an amount of a precipitant;
comparing the amount of precipitant with the at least one measurement to determine a tendency of the at least one quench oil to produce fouling;
selecting a quench oil from a plurality of quench oils; wherein the selected quench oil has the smallest tendency for fouling; and
quenching a hydrocarbon feed with the selected quench oil during a cracking process.

2. The method of claim 1 further comprising adjusting process conditions in the cracking process based upon the tendency of quenching oil in the quenching step to cause fouling.

3. The method of claim 1, further comprising measuring at least one parameter of the quench oil prior to selecting the quench oil, wherein the at least one parameter is selected from the group consisting of an insolubility number, a solubility blending number, and combinations thereof.

4. The method of claim 3, wherein the at least one parameter is monitored over time.

5. The method of claim 1, wherein the fouling is selected from the group consisting of coke fouling, asphaltene fouling, polynuclear aromatic hydrocarbon fouling, coke precursor fouling, and combinations thereof.

6. The method of claim 1, further comprising introducing an antifoulant to the hydrocarbon feed in an effective amount to further reduce fouling from the quench oil.

7. The method of claim 1, wherein the quench oil is selected from the group consisting: of crude oil; the precursors of naphthalene, phenanthrene, pyrene, quinoline, and hydroquinone; alkyl derivatives of naphthalene, phenanthrene, pyrene, quinoline, and hydroquinone; and mixtures thereof.

8. The method of claim 1 wherein the quench oil is selected from the group consisting of: steam cracked quench oils; steam cracked tars; cat cracked tars; cat cracked cycle oils; cat cracked bottoms; coker gas oils; coal tar oils; aromatic extent oils; cuts of steam cracked quench oils, steam cracked tars, cat cracked tars, cat cracked cycle oils, cat cracked bottoms, coker gas oils, coal tar oils, and aromatic extract oils; and mixtures thereof.

9. The method of claim 1 wherein the hydrocarbon feed is used to produce ethylene, gasoline, diesel fuel, other fuel oils, or coke.

10. The method of claim 9 wherein the hydrocarbon feed is used to produce ethylene.

11. The method of claim 1 further comprising introducing aliquots of a precipitant to the quench oil and measuring the light scattering properties of the quench oil.

12. The method of claim 11, wherein a quench oil candidate requiring more precipitant to increase light scattering is considered less likely to foul than a quench oil candidate requiring less precipitant.

13. The method of claim 12, wherein the precipitant is selected from the group consisting of pentane, hexane, heptane, octane, isobutane, cyclohexane, and mixtures thereof.

14. The method of claim 1, wherein the probe is a fiber optic probe.

15. The method of claim 1 further comprising using solvent to dilute the quench oil prior to measuring the tendency of the quench oil to precipitate polymeric species.

16. A method for reducing coke-fouling from quench oil comprising:
obtaining at least one measurement related to light scattering properties for at least one quench oil with a probe; wherein the at least one quench oil comprises an amount of a precipitant;
comparing the amount of precipitant with the at least one measurement to determine a tendency of the at least one quench oil to produce coke-fouling;
selecting a quench oil from a plurality of quench oils; wherein the selected quench oil has the smallest tendency for coke-fouling; and
quenching a hydrocarbon feed with the selected quench oil during a cracking process.

17. The method of claim 16, further comprising measuring at least one parameter of the quench oil, wherein the at least one parameter is selected from the group consisting of an insolubility number, a solubility blending number, and combinations thereof.

18. The method of claim 17, wherein the at least one parameter is monitored over time.

19. The method of claim 16, wherein the fouling is selected from the group consisting of coke fouling, asphaltene fouling, polynuclear aromatic hydrocarbon fouling, coke precursor fouling, and combinations thereof.

20. The method of claim 17, wherein a quench oil candidate requiring more precipitant to increase light scattering is considered less likely to foul than a quench oil candidate requiring less precipitant.
